(19) United States
(12) Patent Application Publication
(10) Pub. No.: US 2008/0287670 A1
(43) Pub. Date: Nov. 20, 2008

(54) Systems and methods for the purification of synthetic trityl-on oligonucleotides

(76) Inventors: Gregory Scott, Hermosa-Beach, CA (US); Avelino Sanchez, Torrance, CA (US); Krishnamohanrao S. Kallury, Torrance, CA (US)

Correspondence Address: STETINA BRUNDA GARRED & BRUCKER, 75 ENTERPRISE, SUITE 250, ALISO VIEJO, CA, 92656, US

(21) Appl. No.: 11/804,538

(22) Filed: May 18, 2007

(51) Int. Cl. C07H 21/04; B01J 39/08; C07H 21/02

(52) U.S. Cl. 536/25.42; 252/60

(57) ABSTRACT

Chromatographic loading solutions for use in the purification of trityl-on oligonucleotides. The solutions comprise an antichaotropic ion, a chaotropic ion, an alkaline salt, and a polar protic solvent, all at particular concentrations. The solution is useful in purifying oligodeoxyribonucleotides and oligoribonucleotides having either ACE or TBDMS protective caps. Also, methods and systems for purifying trityl-on oligonucleotides comprising the chromatographic loading solution, a reversed-phase sorbent, and the oligonucleotides to be purified.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

Once utilized as only molecular research tools for gene amplification and detection, synthetic oligonucleotides are rapidly becoming staples in diagnostic and therapeutic applications. The recent discoveries of unsuspected oligonucleotide function have launched these molecules into the forefront as potential treatments in nearly every area of medicine notably, oncology, ophthalmology, neurology and immunology. Although pharmacological breakthroughs will take years or even decades to reach commercial status, oligonucleotide chemistry has been and will continue to be the corner stone of life science research. Contemporary findings involving RNA and DNA chemistries have provided astounding progression in not only drug development, but in a host of other vital areas such as system biology, cellular pathways, gene mapping, and gene function analysis. The monetary expectation for the oligonucleotide market is substantial. According to most analyst projections, the fledging RNAi market alone will double to over $1 billion by 2010. Coinciding with modern advances is the continued growth of the decades old PCR market as the advent of microarrays fuels the demand for parallel analysis techniques. While the progression of novel and proven applications of oligonucleotides is very much welcomed by synthetic oligo producers, it comes with a host of new requirements and restrictions. To meet the rigors of heavily regulated markets, oligo manufacturers are addressing a growing burden to provide higher purities and greater yields of their synthetic products. Furthermore, as end-users alter their experimentations from in-vitro to in-situ designs, synthetic impurities once permissible for enzyme-based assays must now be removed to accommodate cellular and tissue-base investigations.

State-of-the-art oligosynthesis utilizes solid-phase phosphoramidite chemistry to construct both oligoribo- and oligodeoxyribonucleotides through a succession of phosphodiester linkages. Advances in synthetic chemistry have introduced a variety of new reagents and synthetic schemes, yet the majority of contemporary oligosynthesis platforms continue to adhere to dimethoxytrityl (DMT) chemistry designed over 40 years ago. Assembled from 3′ to 5′, the typical oligosynthesis cycle encompasses four automated steps, detritylation, coupling, capping, and oxidation. Abiding to the traditional approach, the initial protected phosphoramidite is linked at the 3′ hydroxyl position to a solid porous support, controlled pore glass bead, (“CPG”). The first step of the elongation cycle removes the acid labile trityl group at the 5′ carbon of the sugar backbone liberating reactive hydroxyl groups for attachment of the subsequent protected phosphoramidite. A coupling reagent, tetrazole, is then added forming a tetrazolyl phosphoramidite intermediate enabling the formation of a phosphodiester linkage at the respective 5′ and 3′ hydroxyls of attaching nucleotides. This is followed by a capping step where an acetylating reagent is added to irreversibly cap unreacted free hydroxyl groups thus limiting unwanted by-products from failed coupling. Oxidation is the fourth and final step with the addition of iodine to stabilize phosphate linkages. After the desired sequence length has been achieved, the crude oligonucleotide is then removed from its solid support in an ammonia solution then heated to remove alkaline-labile protecting groups from the nucleobases and phosphate backbone. Synthetic ribonucleotides, however, require a more gentle cleavage process, as these molecules are not stable in caustic solutions nor do they tolerate elevated temperatures.

Throughout the assembly process, sequence fragments and other synthetic contaminants are introduced and diminish the yield of the full-length oligo product.

For DNA, modern oligosynthesis coupling failure rates should be no greater than 0.5% per coupling event. Thereby, the cumulative population failures of a given sequence can be estimated from the following equation: xⁿ, wherein x is the success percentage rate and n is number of couplings. For example, synthesis of a 21-mer sequence on an instrument providing 99.5% efficiency [(0.995)²⁰] would yield 90.5% of full-length product. The remnant impurities constitute the non-full-length mass of the total yield, which consist primarily of truncated sequences resulting from errant coupling and inefficient detritylation.

RNA solid-phase chemistry uses a similar assembly process as DNA; however, an important distinction between the two chemistries is the added protecting group at the 2′ hydroxyl group on the pentose sugar of ribo-phosphoramidites. As such, RNA construction requires an intricate synthetic design that secures 2′ hydroxyl protection while removing 5′ hydroxyl protection necessary for sequence elongation. Commercially available RNA synthetic schemes are limited to the 5′ dimethyltrityl (DMT) and 2′ t-butyldimethylsilyl chemistries. This approach suffers from diminished coupling efficiencies, often only 96-98%, while also introducing such adverse reactions as 2′-3′ isomerization. Consequently, following this synthetic scheme, ribonucleotides contain considerably more impurities than its DNA counterpart. Although proprietary, novel orthoesters have been developed for 2′ hydroxyl protection that provide significant improvement in RNA synthetic yields and purity. Recognized as ACE chemistry, the advanced synthetic platform constructs full-length RNA polymers in yields comparable to synthetic DNA sequences.

Various purification techniques are available for removing contaminants from crude synthetic oligonucleotides. Like advances in oligo synthetic chemistries, improvements have been made in the purification process. Solvent precipitation once the mainstay has become obsolete through advances in chromatographic resins and automated systems. Gel filtration sorbents composed of cross-linked dextran were one of the first chromatographic methods introduced for removing synthetic by-products such as reagents and salts. Referred to as desalting, the technique is still routinely used for purification of both crude and purified oligonucleotides. Provided in various formats from large-scale preparative systems to small-volume disposable cartridges, desalting is quite effective for eliminating low molecular impurities, but lacks the necessary capacity to remove the more tenacious-bound truncated sequences from full-length products. Furthermore, desalting resins cannot tolerate extreme acidic or caustic solutions requiring the added step of solvent switching.

Contemporary oligo purification requires mass-scale processes. Today, synthetic oligonucleotides are predominately purified with HPLC techniques using either trityl-on or trityl-off methodologies. Those employing trityl-on procedures rely primarily on reversed-phase chromatography, while IEX (ion exchange chromatography) is the mode of choice for the trityl-off contingent. The two chromatographic modalities come with their particular advantages and disadvantages.

By far, IEX provides the highest level of purity of the two chromatographic schemes. Utilizing selective ionic interactions between an oligo analyte and a suitable sorbent, a full-length sequence product can be eluted free of virtually all synthetic impurities in an aqueous salt-saturated medium. Furthermore, trityl-on applications may also be used with the IEX mode yielding exceptional purities of synthetic oligo analytes. While certainly efficacious, there are serious downsides with the ion exchange mode for oligo purification. Particularly, IEX has very low capacity, thus making the technique prohibitive for large-scale purification. Moreover, the technique cannot be easily automated, thus making it quite laborious and unfeasible for multiplex formats. Finally, down-stream desalting purification is required thereby further increasing the total process time and ultimately costs.

The alternative, reversed-phase chromatography separates based upon the hydrophobic properties of an analyte and is practiced exclusively with trityl-on oligo purification. Through differences in hydrophobicity, the trityl-on full-length sequence can be isolated from remnant trityl-off impurities. Moreover, elutions are carried-out in a salt-free organic solvent, thus eliminating down-stream desalting purification. Unlike IEX, reversed-phase techniques can be easily automated and offer improved loading capacity. While more efficient than IEX, reversed-phase separation does have significant drawbacks, notably purity. Truncated sequences, particularly failed tritylated sequences, have the same chromatographic mobility as the full-length sequence product and cannot be resolved under reserved-phase conditions. In addition, optimal RP-HPLC resolution requires ion-pairing agents for enhancing hydrophobic separation. These agents are not only quite toxic but are rather difficult to remove as they bind tenaciously to both the oligo analyte and chromatographic sorbent. Unfortunately, such additives not only reduce the lifetime of the resin, but also for complete removal from the oligonucleotide, require the painstaking task of sequential precipitations or lyophilizations.

Trityl-on cartridge purification was introduced soon after solid phase automation became the synthetic mainstay. Originally designed to alleviate the shortfalls of sequential HPLC purification, cartridge-based products were to function as cost effective alternatives that were fast, efficacious, and conducive for serial purification platforms. To accommodate such features, trityl-on cartridges utilized reversed-phase methodology, thus relying on ion pairing agents, aprotic solvents and gradient elutions. Unfortunately, these products have and continue to fail at providing generally acceptable purity and recovery yields. Moreover, nearly every commercial reversed-phase cartridge product lacks the convenience of direct loading in ammonia-based solutions, thus limiting their utility for continuous serial production. Consequently, for many in the art, cartridge purification has failed to deliver both the conveniences and efficacies as promised.

The problems existing with modern cartridge products lie not necessarily with the resin itself but rather with the accompanying solvent system. The sorbent that is housed in the typical cartridge is of polymeric material, generally a polystyrene derivative and, unlike a dextran or silica-based media, can tolerate both acidic and caustic solutions. Moreover, the properties of the polymeric resin allow for greater hydrophobic retention of a full-length trityl-on oligonucleotide. Mired with conventional reversed-phase wisdom, trityl-on cartridges are coupled with saturated solutions of ion-pairing agents, typically triethylamine acetate (TEAA). Such solutions are added to the resin prior to loading to augment the retention of a full-length 5′-protected oligonucleotide. However, when a synthetic trityl-on oligo is administered to a commercial polymer cartridge in an ammonia-based cocktail, much of the full-length product is not retained on the sorbent and is ultimately lost during the loading step. This results from inefficient reagent composition of the loading solutions of commercially available products. Even with ion pairing, a three-fold water dilution of the ammonia solution is generally performed before the analyte is applied to the cartridge. Most current products also recommend collecting and re-administering the load fraction to ensure optimal retention of the full-length trityl-on sequence. This common practice of repetitive loading cycles results in sample loss and adversely affects the recovery yield of the final product. The added ion-pairing agent creates further complications as unwanted truncated impurities, commonly trityl-off sequences, become bound to the resin with the full-length product. Since there is no complete discrimination between protected and unprotected sequences, cartridge products require the delicate balance of multiple washes in dilute acetonitrile to selectively remove trityl-off contaminants while retaining the protected full-length sequence. Such techniques are inherently flawed as aprotic solvents disrupt pi-pi interactions of the 5′ dimethoxytrityl group resulting in co-elution of the trityl-on full-length sequence with truncated impurities during sequential wash steps. In addition to low final product recovery, commercial cartridge systems provide purity yields comparable to that of desalting. Clearly, an innovative cartridge purification scheme is warranted and, if efficient and efficacious, such a product would generate significant demand from nearly every segment of the synthetic oligonucleotide market.

### BRIEF SUMMARY

Recent advances in biotechnology research have created a dramatic expansion for synthetic oligonucleotides. The perpetual changing environment for synthetic nucleotides and their mission has demonstrated that there is an urgent need for a more efficient, and efficacious purification platform that matches the need of manufacturers and their customers. The following introduces a new cartridge purification product that will better complement modern synthetic processes and utilization of oligonucleotides.

The present invention relates to the cartridge purification of synthetic trityl-on oligonucleotides. Addressing the global aim of a purification process, the invention delivers concentrated full-length oligo sequences free of impurities in a stable media suitable for in-vivo applications. Simple in practice and in theory, the invention offers speed and efficacy in formats that can be readily automated and suitable for both combinatorial-scale and large-scale purifications. The invention comprises only the formulas of biological compatible agents that when used with a reversed-phase resin significantly enhances the retention mechanism of trityl-on oligonucleotides. Unique to the invention is the capacity to provide greater proficiency when mixed directly with caustic solutions used for cleavage and deprotection in the synthetic process.

Encompassing the formulas are antichaotropic and chaotropic ions, polar protic solvents, and alkaline salts. Following proper conditioning of a polymeric reversed-phase resin, a simple three step process of load, detritylation, and elution is all that is required to obtain a concentrated and highly purified full-length oligo sequence. For synthetic DNA and RNA ACE chemistry, the process begins after an equal volume of the invented formula is mixed with an ammonia based cleavage solution. Next, the solubilized crude oligo analyte is then passed through the reversed-phase sorbent that is housed in either an individual cartridge or multi-well plate. Utilizing the principles of hydrophobic interaction chromatography (HIC), the invented formulas provide selective retention of the full-length trityl-on sequence, while synthetic impurities such as trityl-off truncated sequences are not retained and are eluted during the initial load stage. Detritylation and elution follow, after which a purified full-length oligonucleotide is recovered. For RNA TBDMS chemistry, the sequential purification steps are consistent with the above description; however, the crude oligo deprotection solution is quenched in an appropriate buffer prior to mixing with an equal volume of the invented formula.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is an HPLC graph indicating the results of DNA Example 1;

FIG. 2 is an HPLC graph indicating the results of DNA Example 2;

FIG. 3 is an HPLC graph indicating the results of RNA Example 1; and

FIG. 4 is an HPLC graph indicating the results of RNA Example 2.

### DETAILED DESCRIPTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

One embodiment of the present invention envisions a chromatography loading solution for use in the purification of synthetic trityl-on oligonucleotides. The solution includes an antichaotropic ion, a chaotropic ion, and an alkaline salt in a polar protic solvent. The antichaotropic ion may be present in a concentration range between about 10 mM and about 5M. The antichaotropic ion may be, but is not limited to, SO₄ and/or Cl ions. The chaotropic ion may also be present in a concentration range between about 10 mM and about 5M. The chaotropic ion may be, but is not limited to, a Na ion. The alkaline salt may be present in a concentration range between about 5 mM and about 100 mM. The alkaline salt may be, but is not limited to, a Na₂CO₃ salt. The polar protic solvent may be, but is not limited to, compounds with the general formula ROH, that may include methanol and those solvents with a hydrogen atom attached to an electronegative atom.

In one embodiment, believed to be particularly well suited for the purification of deoxyribo-oligonucleotides, the chromatography loading solution has the formula of 10 mM Na₂CO₃ and 3M NaCl in a 20% methanol solution. In another embodiment, believed to be particularly well suited for the purification of ribo-oligonucleotides, the chromatography loading solution has the formula of 10 mM Na₂CO₃ and 75 mM Na₂SO₄ in a 20% methanol solution.

The chromatography loading solution of the present invention may be mixed with an ammonia-based cleavage solution containing solublized synthetic trityl-on oligonucleotides. The ammonia-based cleavage solution and the chromatography loading solution may be mixed in equal volumes; likewise, when the oligonucleotides to be purified are ribonucleotides including tert-butyldimethylsilyl (TBDMS) orthoester protecting groups, an equal volume of the chromatography loading solution can be mixed with a quenched deprotecting solution, such as, for example, 1.5M NH₄HCO₃.

The present invention further contemplates a method of purifying synthetic trityl-on oligonucleotides using the above-described loading solution. The method includes mixing the chromatography loading solution with an ammonia-based cleavage solution. This mixture is then used to solublize the synthetic trityl-on oligonucleotides to be purified. The solution, including the oligonucleotides, is then passed through a reversed-phase sorbent, whereby the desired oligonucleotides bind to the sorbent. The oligonucleotides are then detritylated and eluted from the sorbent. This method may be utilized to purify oligodeoxyribonucleotides, oligoribonucleotides using bis(2-acetoxyethoxy)methyl (ACE) orthoester protecting groups, and/or oligoribonucleotides using tert-butyldimethylsilyl (TBDMS) ester protecting groups.

When the method is utilized for purification of oligoribonucleotides having tert-butyldimethylsilyl (TBDMS) ester protecting groups, the 2′ deprotecting solution should be quenched in a buffer prior to mixing with the chromatography loading solution. Suitable buffers may include 1.5M NH₄HCO₃. The reversed-phase sorbent utilized in the method may be housed within a cartridge or within a multi-well plate.

The invention also contemplates a system for purifying synthetic trityl-on oligonucleotides. The system includes a chromatography loading solution, as described above, mixed with an ammonia-based cleavage solution, the synthetic trityl-on oligonucleotides to be purified, and a reversed-phase sorbent. The reversed-phase sorbent may be housed within a cartridge or within a multi-well plate.

The chromatography loading solution, method, and system of the present invention can selectively retain full-length trityl-on synthetic deoxy-oligonucleotides as well as full-length trityl-on ribo-oligonucleotides on reversed-phase resins without retaining truncated impurities. The invention also provides complete discrimination of trityl-on full-length sequences from trityl-off impurities in the presence of ammonia-based aqueous solutions and TBDMS cleavage solutions. The present invention further eliminates the need for sequential washing as is required in prior art reversed-phase cartridge purifications. Further, it has been discovered that the components of the chromatography loading solution of the present invention have a synergistic effect and perform effectively only when combined in the concentrations disclosed of each and not as individual agents. The present invention also allows for one-step loading of DNA and RNA in synthetic cocktails, thus eliminating the practice of recycling of the loading solution through the sorbent as is required in prior art methods. The present invention is capable of retaining select trityl-on analytes in the absence of ion pairing agents on reversed-phase resins and provides efficacious cleaning in the absence of aprotic solvents. As such, the present invention provides many benefits over the prior art purification methods.

The following examples show the efficacy in utilizing the chromatographic loadings solutions of the present invention for obtaining highly purified oligonucleotides in an efficient manner. All of the oligonucleotide samples of the following examples were solubilized in particular chromatographic loading solutions of the present invention and the crude trityl-on portions, load portions, and final elution portions were measured via HPLC.

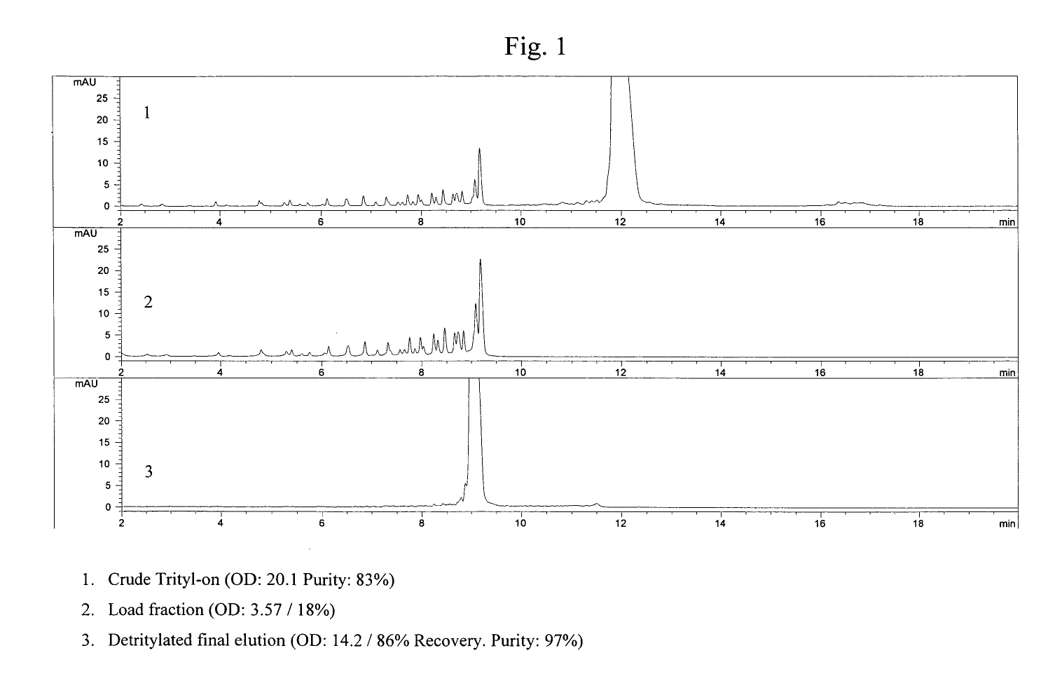

The HPLC process utilized a DNA Pac 200 4×250 mm column. Three mobile phases were utilized comprising water, 0.25 M Tris-HCl at pH 8, and 0.375 M NaClO₄ at gradients of 80%, 10% and 10%-65% over 20 minutes, respectively. The process was run at a flow rate of 1.2 mL/min and UV absorption was measured in the ultraviolet range at 260 nm. The crude trityl-on portion was diluted at a ratio of 1:10 in water (50 μL /450 μL) and a 100 μL sample was injected onto the column. The load portion consisted of the post-cartridge volume collected and a 100 μL sample was injected onto the column. The final elution portion consisted of a 100 μL sample of the total volume and was diluted at a ratio of 1:10 in water (100 μL/900 μL) and a 100 μL sample of this dilution was injected onto the column. The results are shown in graph form in FIGS. 1-4.

### DNA EXAMPLE 1

A trityl-on DNA 24-mer with a molecular weight of 7272.8 was cleaved from its support using methods known within the art and deprotected in 300 μL of concentrated NH₄OH. An equal volume of a chromatographic loading solution comprising 10 mM Na₂CO₃ and 3M NaCl in a 20% methanol solution was added to the deprotected oligonucleotide and the combined volume was mixed by vortexing.

The sample was purified utilizing a 12 position vacuum manifold. 50 mg of a polymeric sorbent was housed within a 1 mL cartridge and conditioned by adding two doses of 0.5 mL of methanol each. A light vacuum was applied to provide a sufficient 2 drops per second solvent flow rate through the media. The sorbent was then equilibrated by adding two doses of 0.5 mL of water each. The vacuum was elevated to ensure a consistent 2 drops per second solvent flow rate through the sorbent.

While the vacuum levels were monitored to ensure consistency, 600 μL of the oligonucleotide solution was gradually administered at a rate of 1 drop per 2-3 seconds through the sorbent. Next, under a moderate vacuum level, 1 mL of aqueous 3% DCA was then added to the sorbent at 1 drop per second to detritylate the oligonucleotide. Then, two doses of 0.5 mL of water each were quickly administered through the sorbent media at a flow rate of 2-3 drops per second. The vacuum was then increased to 10″ Hg to dry the sorbent for approximately 1 minute. The final detritylated product was then slowly eluted at 1 drop per second in 1 mL of buffer comprised of 20 mM NH₄HCO₃/40% Acetonitrile. The crude trityl-on portion, the load portion, and the final elution portion were all collected and subjected to HPLC, as described above. The results are shown in FIG. 1.

### DNA EXAMPLE 2

A trityl-on DNA 30-mer with a molecular weight of 9206 was cleaved from its support using methods known within the art and deprotected in 300 μL of concentrated NH₄OH. An equal volume of a chromatographic loading solution comprising 10 mM Na₂CO₃ and 3M NaCl in a 20% methanol solution was added to the deprotected oligonucleotide and the combined volume was mixed by vortexing.

The sample was purified utilizing a 96-well plate manual vacuum manifold. 50 mg of a polymeric sorbent was housed within each well of the 96-well plate and conditioned by adding two doses of 0.5 mL of methanol to each. A light vacuum was applied to provide a sufficient 2 drops per second solvent flow rate through the media. The sorbent was then equilibrated by adding two doses of 1 mL of water each. The vacuum was elevated to ensure a consistent 2 drops per second solvent flow rate through the sorbent.

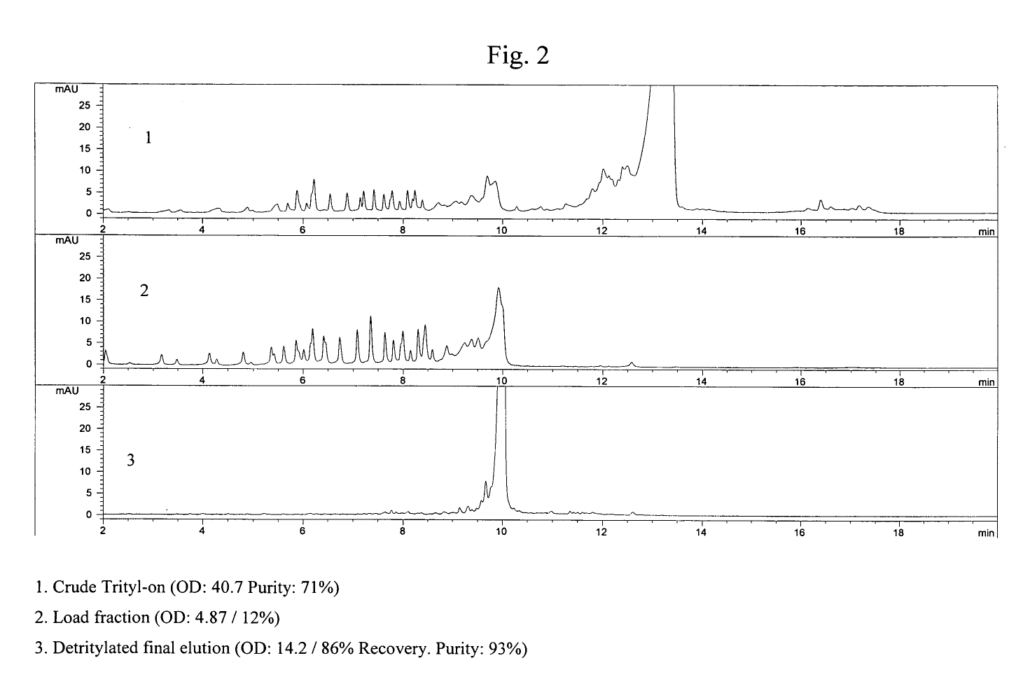

While the vacuum levels were monitored to ensure consistency, 600 μL of the oligonucleotide solution was gradually administered at a rate of 1 drop per 2-3 seconds through the sorbent. Next, under a moderate vacuum level, 1 mL of aqueous 1% DCA was then added to the sorbent at 1 drop per second to detritylate the oligonucleotide. Then, two doses of 0.5 mL of water each were quickly administered through the sorbent media at a flow rate of 2-3 drops per second. The vacuum was then increased to 10″ Hg to dry the sorbent for approximately 1 minute. The final detritylated product was then slowly eluted at 1 drop per second in 1 mL of buffer comprised of 20 mM NH₄HCO₃/20% Acetonitrile. The crude trityl-on portion, the load portion, and the final elution portion were all collected and subjected to HPLC, as described above. The results are shown in FIG. 2.

### RNA EXAMPLE 1

A trityl-on RNA 28-mer with a molecular weight of 8861 was cleaved from its support using methods known within the art and deprotected in 300 μL of a EtOH/NH₄OH (1:3) deprotecting cocktail. The deprotecting cocktail was then entirely removed via evaporation using N₂. The resulting RNA pellet was reconstituted in 100 μL of a silyl deprotecting solution composed of 46% N-methylpyrrolidinone, 23% triethylamine, and 31% triethylamine trihydrofluoride and heated to 65° C. for 1.5 hours. The reaction was quenched by slowly adding 400 μL of NH₄HCO₃. To this solution an equal volume of 500 μL of chromatography loading solution comprising 10 mM Na₂CO₃ and 75 mM Na₂SO₄ in a 20% methanol solution was added and mixed by vortexing.

The sample was purified utilizing a 96-well plate manual vacuum manifold. 50 mg of a polymeric sorbent was housed within each well of the 96-well plate and conditioned by adding two doses of 0.5 mL of methanol to each. A light vacuum was applied to provide a sufficient 2 drops per second solvent flow rate through the media. The sorbent was then equilibrated by adding two doses of 1 mL of water each. The vacuum was elevated to ensure a consistent 2 drops per second solvent flow rate through the sorbent.

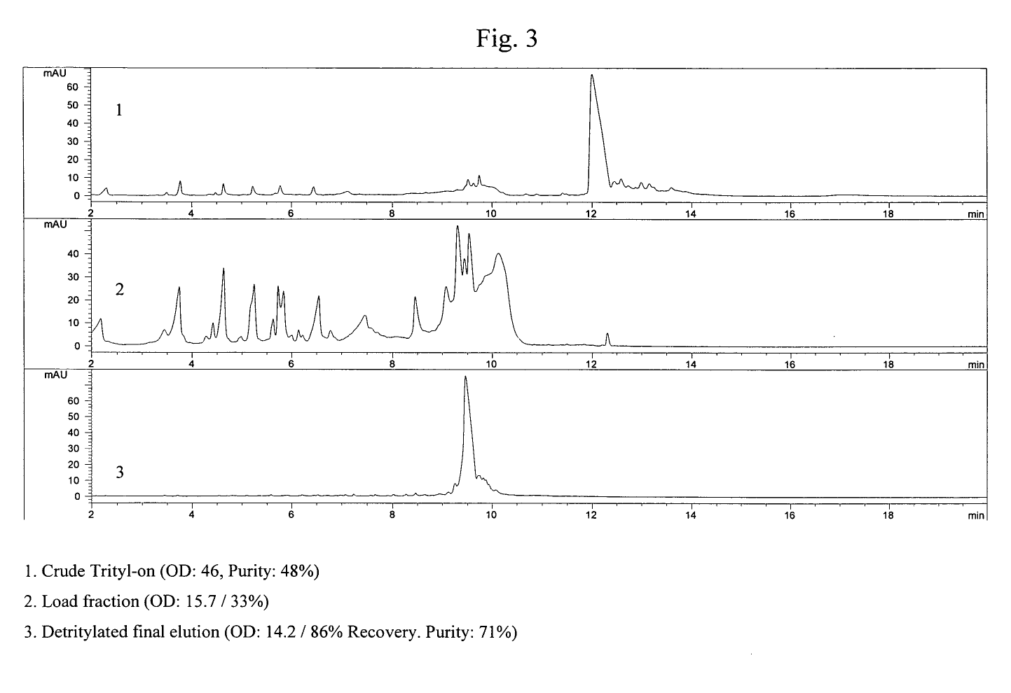

While the vacuum levels were monitored to ensure consistency, 1 mL of the oligonucleotide solution was gradually administered at a rate of 1 drop per 2-3 seconds through the sorbent. Next, under a moderate vacuum level, 1 mL of aqueous 1% DCA was then added to the sorbent at 1 drop per second to detritylate the oligonucleotide. Then, two doses of 0.5 mL of water each were quickly administered through the sorbent media at a flow rate of 2-3 drops per second. The vacuum was then increased to 10″ Hg to dry the sorbent for approximately 1 minute. The final detritylated product was then slowly eluted at 1 drop per second in 1 mL of buffer comprised of 20 mM NH₄HCO₃/20% Acetonitrile. The crude trityl-on portion, the load portion, and the final elution portion were all collected and subjected to HPLC, as described above. The results are shown in FIG. 3.

### RNA EXAMPLE 2

A trityl-on RNA 21-mer with a molecular weight of 6221 was cleaved from its support using methods known within the art and deprotected in 1 mL of a EtOH/NH₄OH (1:3) deprotecting cocktail. The deprotecting cocktail was then entirely removed via evaporation using N₂. The resulting RNA pellet was reconstituted in 700 μL of a silyl deprotecting solution composed of 46% N-methylpyrrolidinone, 23% triethylamine, and 31% triethylamine trihydrofluoride and heated to 65° C. for 1.5 hours. The reaction was quenched by slowly adding 800 μL of NH₄HCO₃. To this solution an equal volume of 1.5 mL of chromatography loading solution comprising 10 mM Na₂CO₃ and 75 mM Na₂SO₄ in a 20% methanol solution was added and mixed by vortexing.

The sample was purified utilizing a 12 position vacuum manifold. 150 mg of a polymeric sorbent was housed within a 3 mL cartridge and conditioned by adding two doses of 1.5 mL of methanol each. A light vacuum was applied to provide a sufficient 2 drops per second solvent flow rate through the media. The sorbent was then equilibrated by adding two doses of 1.5 mL of water each. The vacuum was elevated to ensure a consistent 2 drops per second solvent flow rate through the sorbent.

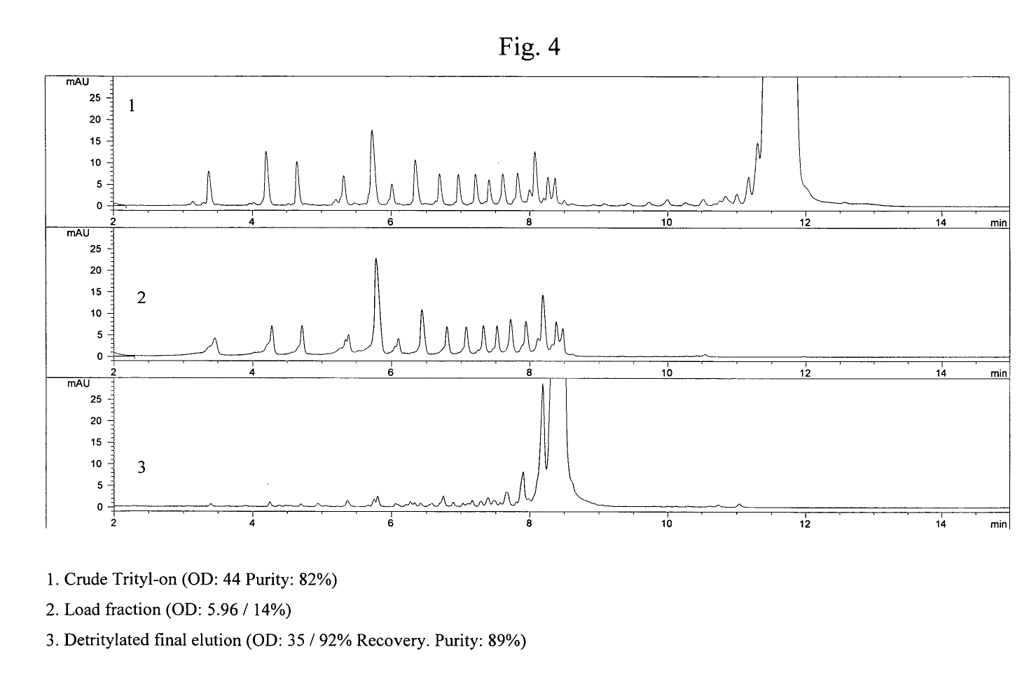

While the vacuum levels were monitored to ensure consistency, 3 mL of the oligonucleotide solution was gradually administered at a rate of 1 drop per 2-3 seconds through the sorbent. Next, under a moderate vacuum level, 1.5 mL of aqueous 3% DCA was then added to the sorbent at 1 drop per second to detritylate the oligonucleotide. Then, two doses of 1 mL of water each were quickly administered through the sorbent media at a flow rate of 2-3 drops per second. The vacuum was then increased to 10″ Hg to dry the sorbent for approximately 1 minute. The final detritylated product was then slowly eluted at 1 drop per second in 2 mL of buffer comprised of 15 mM Na₂CO₃/40% Acetonitrile. The crude trityl-on portion, the load portion, and the final elution portion were all collected and subjected to HPLC, as described above. The results are shown in FIG. 4.

Table 1 shows the efficacy of utilizing the present invention and indicates the level of purity obtainable from a single pass-thru utilizing the chromatography loading solutions, methods, and systems of the present invention. In particular, the optical density of crude samples, load samples, and detritylated final elutions were measured at a wavelength of 260 nm. The crude samples and detritylated final elutions were diluted at a ratio of 1:100, whereas the load samples were not diluted.

TABLE 1

| | | | | | | | Purified | | | | |
|---|---|---|---|---|---|---|---|---|---|---|---|
| | | | | | | | | | | % | |
| | | | | | | | | | | Recovery | |
| | | Crude | | | | | | | | Final/ | Purity |
| | Mol | | | | Load | Crude- | Final | | | (Crude- | (% |
| Length | Weight | OD_{260(1/100)} | mg/mL | nmoles | OD₂₆₀ | Load_{(OD}) | OD_{260(1/100)} | mg/mL | nmoles | Load) | Area) |
| 21 | 6397.3 | 0.171 | 0.56 | 88.2 | 3.16 | 13.94 | 0.118 | 0.39 | 60.9 | 84.6% | 97% |
| 22 | 6695.4 | 0.194 | 0.64 | 95.6 | 3.26 | 16.14 | 0.139 | 0.46 | 68.5 | 86.1% | 95% |
| 24 | 7272.8 | 0.201 | 0.66 | 91.2 | 3.57 | 16.53 | 0.142 | 0.47 | 64.4 | 85.9% | 97% |
| 39 | 11,982.9 | 0.417 | 1.38 | 114.8 | 5.99 | 35.71 | 0.332 | 1.10 | 91.4 | 93.0% | 87% |
| 43 | 13,119.6 | 0.438 | 1.45 | 110.2 | 6.45 | 37.36 | 0.341 | 1.13 | 85.8 | 91.3% | 85% |
| 53 | 16,221.7 | 0.397 | 1.31 | 80.8 | 6.51 | 33.19 | 0.296 | 0.98 | 60.2 | 89.2% | 93% |
| 56 | 16,971.2 | 0.48 | 1.58 | 93.3 | 6.65 | 41.35 | 0.37 | 1.22 | 71.9 | 89.5% | 90% |

The invented formulas can be used for any synthetic single-stranded oligonucleotide regardless of length or synthetic derivatization and or adjunct. Furthermore, the formulas are compatible with the standard TBDMS RNA chemistry as well as the modern ACE RNA chemistry. While polar protic solvents and alkaline salts may remain constant in the formulas, chaotropic and antichaotropic agents may be varied for optimal purification of deoxyribo- and ribo-oligonucleotides.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A chromatography loading solution for use in the purification of synthetic trityl-on oligonucleotides, the solution comprising:
antichaotropic ions in the concentration range of about 10 mM to about 5M;
chaotropic ions in the concentration range of about 10 mM to about 5M;
an alkaline salt in the concentration range of about 5 mM to about 100 mM; and
a polar protic solvent.

2. The chromatography loading solution of claim 1, wherein the antichaotropic ions are Cl⁻ ions.

3. The chromatography loading solution of claim 2, wherein the Cl⁻ ions are at a concentration of 3M.

4. The chromatography loading solution of claim 1, wherein the antichaotropic ions are SO₄⁻² ions.

5. The chromatography loading solution of claim 4, wherein the SO₄⁻² ions are at a concentration of 75 mM.

6. The chromatography loading solution of claim 1, wherein the chaotropic ions are Na⁺ ions.

7. The chromatography loading solution of claim 3, wherein the chaotropic ions are Na⁺ ions at a concentration of 3M.

8. The chromatography loading solution of claim 5, wherein the chaotropic ions are Na⁺ ions at a concentration of 75 mM.

9. The chromatography loading solution of claim 1, wherein the alkaline salt is Na₂CO₃.

10. The chromatography loading solution of claim 9, wherein the alkaline salt is present in the solution at a concentration of 10 mM.

11. The chromatography loading solution of claim 1, wherein the polar protic solvent is methanol.

12. The chromatography loading solution of claim 11, wherein the polar protic solvent is 20% methanol.

13. The chromatography loading solution of claim 1 further comprising an ammonia-based cleavage solution.

14. The chromatography loading solution of claim 13, wherein the chromatography loading solution and the ammonia-based cleavage solution are mixed in equal volumes.

15. The chromatography loading solution of claim 13, wherein the ammonia-based cleavage solution is diluted with water or a buffer prior to mixing with the chromatography loading solution.

16. A chromatography loading solution for use in the purification of synthetic trityl-on deoxyribo-oligonucleotides, the solution comprising:
NaCl in the concentration range of about 10 mM to about 5M;
Na₂CO₃ in the concentration range of about 5 mM to about 100 mM; and
a polar protic solvent.

17. The chromatography loading solution of claim 16, wherein the NaCl is at a concentration of 3M, the Na₂CO₃ is at a concentration of 10 mM, and the polar protic solvent is 20% methanol.

18. A chromatography loading solution for use in the purification of synthetic trityl-on ribo-oligonucleotides, the solution comprising:
Na₂SO₄ in the concentration range of about 10 mM to about 5M;
Na₂CO₃ in the concentration range of about 5 mM to about 100 mM; and
a polar protic solvent.

19. The chromatography loading solution of claim 18, wherein the Na₂SO₄ is at a concentration of 75 mM, the Na₂CO₃ is at a concentration of 10 mM, and the polar protic solvent is 20% methanol.

20. A method of purifying synthetic trityl-on oligonucleotides comprising the steps:
a) mixing a chromatography loading solution comprising an antichaotropic ion, a chaotropic ion, an alkaline salt, and a polar protic solvent with an equal volume of an ammonia-based cleavage solution;
b) solubilizing the synthetic trityl-on oligonucleotides in the solution of step a);
c) passing the solution of step b) through a reversed-phase sorbent;
d) detritylating the oligonucleotides; and
e) eluting the oligonucleotides.

21. The method of claim 20, wherein the synthetic trityl-on oligonucleotides comprise deoxyribonucleotides.

22. The method of claim 20, wherein the synthetic trityl-on oligonucleotides comprise ribonucleotides having bis(2-acetoxyethoxy)methyl orthoesters.

23. The method of claim 20, wherein the synthetic trityl-on oligonucleotides comprise ribonucleotides having tert-butyldimethylsilyl esters.

24. The method of claim 23, wherein the ammonia-based cleavage solution in step a) is quenched prior to mixing in equal parts with the chromatography loading solution.

25. The method of claim 24, wherein the ammonia-based cleavage solution in step a) is a 1.5M NH₄HCO₃ solution.

26. The method of claim 20, wherein the reversed-phase sorbent is housed within a cartridge.

27. The method of claim 20, wherein the reversed-phase sorbent is housed within a multi-well plate.

28. A system for purifying synthetic trityl-on oligonucleotides comprising:
chromatography loading solutions comprising an antichaotropic ion, a chaotropic ion, an alkaline salt, and a polar protic solvent mixed with an equal volume of an ammonia-based cleavage solution;
synthetic trityl-on oligonucleotides; and
a reversed-phase sorbent.

29. The system of claim 28, wherein the ammonia-based cleavage solution is a 2′ quenched deprotecting solution.

30. The system of claim 29, wherein the 2′ quenched deprotecting solution is a 1.5M NH₄HCO₃ solution.

31. The system of claim 28, wherein the reversed-phase sorbent is housed within a cartridge.

32. The system of claim 28, wherein the reversed-phase sorbent is housed within a multi-well plate.
